(19) United States
(12) Patent Application Publication
(10) Pub. No.: US 2023/0026497 A1
(43) Pub. Date: Jan. 26, 2023

(54) Container Having Reinforced Sealing Properties

(71) Applicant: ABC TRANSFER, Tours (FR)

(72) Inventors: Julien FELIX, Tours (FR); Jean-Luc SCHNEIDER, Sligo (FR); Thierry GIRARD, Tours (FR)

(21) Appl. No.: 17/787,505

(22) PCT Filed: Dec. 17, 2020
(86) PCT No.: PCT/FR2020/052499

§ 371 Date: Jun. 20, 2022

(30) Foreign Application Priority Data
Dec. 20, 2019 (FR) FR1915253

(51) Int. Cl. B65D 53/02; B65D 45/32; B65D 81/18

(52) U.S. Cl. CPC B65D 53/02; B65D 45/322; B65D 81/18; B65D 2251/205

(57) ABSTRACT

The invention relates to a sealed container (1) comprising a flange (5, 6) that delimits a through-passage having an axial axis in the container, a door (7) movably mounted on the flange (5, 6), and a first annular seal (13) located between the flange (5, 6) and the door (7), the flange, the door and the first seal forming a sealed connection device that can be connected to a complementary connection device on a sterile enclosure by bayonet connection means in order to allow sterile communication between the sealed container and the sterile enclosure, characterized in that the container (1) includes a protective cover (20) on the connection device, said protective cover being forcibly clipped onto the container, and in that the container further includes contact pressure means arranged between the protective cover (20) and the connection device so as to apply contact pressure to the connection device in the axial direction proportional to the clipping force.

### TECHNICAL FIELD OF THE INVENTION

The invention relates to the field of containers intended to be connected in a sealed manner to sterile enclosures so as to allow their respective volumes to be placed in communication without contact with said external environment.

The invention relates more particularly to a sealed container comprising a flange that delimits a through-opening having an axial axis in the container, a door movably mounted on the flange and a first annular seal located between the flange and the door, the flange, the door and the first seal forming a sealed connection device that can be connected to a complementary connection device on a sterile enclosure by bayonet connection means in order to allow sterile communication between the sealed container and the sterile enclosure.

The sealed container according to the invention is intended in particular, but not exclusively, for the transfer of hazardous products such as certain pharmaceutical, biotechnological, biological, chemical or radioactive products, the transfer of components such as stoppers, bottles, plungers, syringes, etc., the transfer of environmental control devices such as culture medium racks, particle counters, etc., the transfer of cleaning systems, the transfer of liquids, powders, tools, the transfer of waste to the outside of the enclosure and/or the transfer of any element necessary for the production or maintenance of the production line.

### PRIOR ART

In a known manner, the connection of a sealed container to a sterile enclosure for the transfer of products, without breaking the connection and the seal, is carried out using two connection devices, one equipping the container and the other equipping the enclosure. Each connection device comprises a flange respectively delimiting the through-opening in the internal space of the container or of the enclosure, each of the through-openings being closed off by a door. The flange and the door of the container are capable of being connected respectively to the flange and to the door of the enclosure by a bayonet connection and separated from one another under the action of a rotational movement of the associated flange and door of the container with respect to the flange and door of the enclosure to which they are attached.

On the whole, the connection devices are satisfactory from a sealing point of view. However, it cannot be excluded that these devices may be damaged during the transport, storage and/or handling thereof and therefore that the sealing of the containers will be altered.

The invention aims to remedy this problem by proposing a sealed container that can be connected to a sterile enclosure making it possible to reinforce the sealing of the container while guaranteeing that the container has not undergone any alteration from a sealing point of view during its transport and/or storage.

### SUBJECT MATTER OF THE INVENTION

To this end, the invention proposes a sealed container comprising a flange that delimits a through-opening having an axial axis in the container, a door movably mounted on the flange and a first annular seal located between the flange and the door, the flange, the door and the first seal forming a sealed connection device that can be connected to a complementary connection device on a sterile enclosure by bayonet connection means in order to allow sterile communication between the sealed container and the sterile enclosure. The container is remarkable in that it comprises a protective cover on the connection device forcibly clipped onto the container and contact pressure means arranged between the protective cover and the connection device so as to apply contact pressure to the connection device in the axial direction proportional to the clipping force.

Advantageously, the contact pressure means are arranged to exert contact pressure on the door.

Provision may also be made for the contact pressure means to be arranged to exert contact pressure on the first seal alone or also on the door.

Advantageously, the protective cover comprises an internal surface provided with an annular boss forming the pressure means. The annular boss may be continuous or discontinuous.

Advantageously, the pressure means are formed by a second annular seal inserted between the protective cover and the connection device.

Advantageously, the container has a container body formed by a flexible film fixed to the flange, said flange being made up of two parts that are separate from each other, respectively forming an external sleeve and an internal sleeve, the flexible film being kept engaged between the external sleeve and the internal sleeve.

Advantageously, the container comprises a ring for clamping the external sleeve on the internal sleeve arranged to grip the parts of the external sleeve and of the internal sleeve between which the flexible film is interposed.

Advantageously, the protective cover comprises flexible tabs extending in the direction of the clamping ring and engaged with the latter in an irreversible manner.

Advantageously, the container comprises a third annular seal housed in an internal peripheral groove of the external sleeve, the flexible film extending between the annular seal and the internal sleeve.

Advantageously, the external sleeve has a lower skirt comprising longitudinal slots extending from the lower end edge of the external sleeve and evenly distributed over the periphery of said sleeve so as to form elastic tabs capable of exerting a clamping force on the internal sleeve.

Advantageously, the flexible film and the connection device are made of a bacteriostatic material.

### BRIEF DESCRIPTION OF THE DRAWINGS

Other features and advantages of the invention will become apparent from the following detailed description of the invention with reference to the appended figures, in which:

FIG. 1 shows a schematic perspective view of a container according to the invention, the protective cover being shown not assembled;

FIG. 1a shows a view of the container of FIG. 1 equipped with the protective cover;

FIG. 2 shows a sectional view of the container of FIG. 1a;

FIG. 3 shows a detail view of the container of FIG. 2;

FIG. 4 shows a partial view of the external sleeve 5 of the connection device of the container of FIG. 2;

FIG. 5 shows another detail view of the container of FIG. 2:

FIG. 6 shows a bottom view of the protective cover of the container;

FIG. 7 shows a detail view of the container of FIG. 2;

FIG. 7a repeats the detail view of FIG. 7, a tab of the protective cover being shown deformed;

FIG. 8 shows a detail view of an alternative embodiment of the container according to the invention.

### DETAILED DESCRIPTION OF THE INVENTION

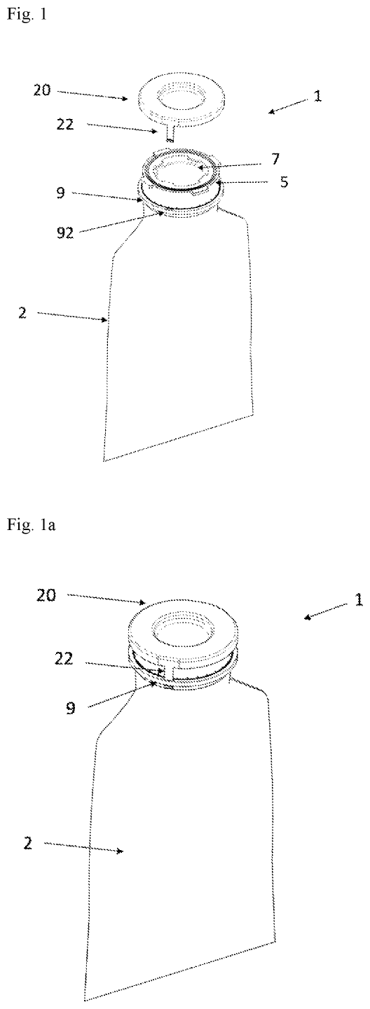

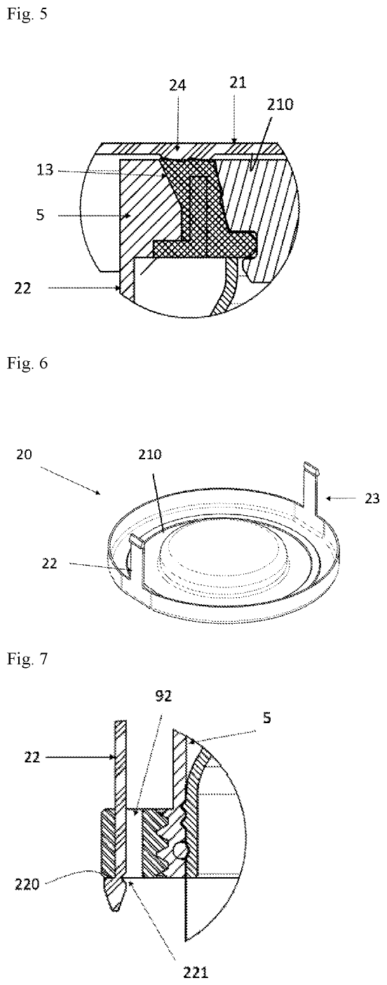

In relation to FIGS. 1 to 6, a container 1 is described according to one embodiment of the invention. The illustrated container 1 comprises a flexible film 2 arranged to define an internal space 3 and a through-opening, defining an axial, allowing the internal space 3 to be placed in communication with the exterior of the film. The container also comprises a sealed connection device that is arranged to allow the flexible film 2 to be connected to a complementary connection device of a sterile enclosure. The connection is made in such a way as to ensure sterile communication between the internal space 3 of said container 1 and that of the enclosure.

The connection device comprises a flange on which the flexible film is attached, said flange delimiting the through-opening of the flexible film. It further comprises a removable door 7 attached to the flange so as to close the through-opening. Advantageously, the flange and the door 7 are attached to one another by means of notches and lugs respectively borne by the flange and the door.

In the illustrated example, the flange is constituted by two mutually different pieces, a first piece forming a so-called external sleeve and a second piece forming a so-called internal sleeve, between which the flexible film extends. The internal sleeve 6 comprises a notched zone 64 adjacent to the bearing recess arranged to cooperate with a complementary notching 54 provided on the inner face 50 of the external sleeve 5. According to an advantageous configuration illustrated in FIG. 4, the external sleeve 5 comprises a lower skirt 10 comprising longitudinal slots 11 extending from the lower end edge of said sleeve. These longitudinal slots 11 are advantageously evenly distributed over the periphery of said sleeve. They thus delimit elastic tabs 12 capable of exerting a uniform clamping force over the entire outer periphery of the internal sleeve 6 when the external sleeve 5 is mounted on the internal sleeve 6. The elastic tabs 12 comprise means of reciprocal association with clamping means, described below, and the internal sleeve 6, respectively. In the example illustrated, the means of reciprocal association respectively with the clamping means and the internal sleeve 6 consist of a thread 53 and a notching 54. The clamping means are in the form of an external clamping ring 9 comprising an internal thread 90 with a shape complementary to the thread 53 provided on the outer face 52 of the external sleeve 5. The clamping means are arranged to grip the parts of the external sleeve 5 and of the internal sleeve 6 between which the flexible film 2 is interposed.

The container 1 further comprises an annular seal 8 housed in a peripheral groove formed on the inner face 50 of the external sleeve 5 and the outer face 60 of the internal sleeve 6. It defines a flange/bag seal. As shown in FIG. 3, the flexible film 2 is kept gripped between the annular seal 8 and the internal sleeve 6. In order to improve the retention of the flexible film 2 between the two sleeves constituting the flange, the internal sleeve 6 comprises an outer face provided with a bearing recess located opposite the peripheral groove of the external sleeve 5. Thus, when the external sleeve 5 is clamped on the internal sleeve 6, the annular seal 8 crushes the flexible film 2 at the bearing recess, ensuring improved engagement of the flexible film 2.

The container 1 further comprises an annular seal 13 that is borne by the flange. It defines a door/flange seal. When the door is in the position closing off the through-opening, the annular seal 13 is housed in the space delimited by the external sleeve 5, the internal sleeve 6 and the door 7. Advantageously, the annular seal 13 has a cross-section of shape matching the area in which it is housed. In the illustrated embodiment, the annular seal 13 has an L-shaped section.

According to the invention, the container comprises a protective cover 20 of the connection device forcibly clipped onto the flange 5, 6. In the illustrated embodiment, the protective cover 20 comprises a covering part 21 placed opposite the front face of the container, at the outer periphery of which two tabs 22, 23 extend in the direction of the clamping ring 9. “Front face” means the face of the container with respect to its connection with the enclosure.

In other words, it is the face of the container brought opposite the enclosure for the connection of said container to the enclosure.

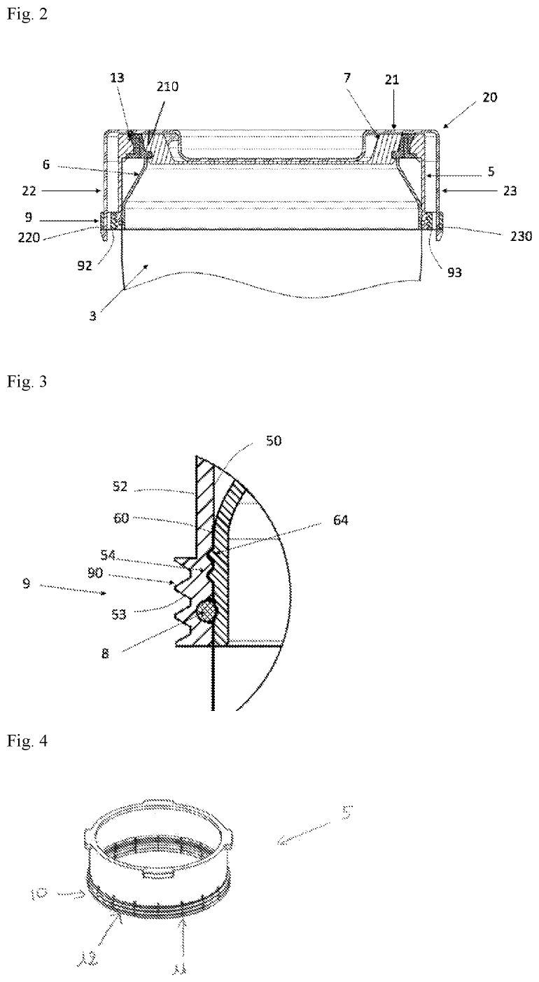

Each tab 22, 23 is provided at its free end with a shoulder 220, 230 formed by a bulge of material that extends, in the illustrated example, radially on the opposite side of the covering part 21. The tabs 22, 23 extend, advantageously substantially parallel to the axial axis, in the direction of the clamping ring 9 and pass respectively through an associated aperture 92, 93 made in said clamping ring 9 so that the shoulders 220, 230 are located under the clamping ring 9. Thus, as shown in FIG. 2, the clipping of the protective cover 20 consists in engaging the shoulders 220, 230 of the tabs 22, 23 on the rear bearing rim of the clamping ring 9 defining the apertures.

In the illustrated embodiment, the protective cover 20 comprises two clipping tabs 22, 23 located diametrically opposite one another. This is an example embodiment; the number and distribution of tabs may vary depending on the dimensions of the opening of the container.

The protective cover 20 further comprises contact pressure means arranged between the protective cover 20 and the connection device so as to exert contact pressure on the first seal 13 in the axial direction proportional to the clipping force. In the illustrated example, the pressure means are formed by an annular boss 24 on the inner face 210 of the covering part 21. The annular boss 24 is thus arranged to be placed facing the door/flange seal 13 when the protective cover 20 is placed on the container. In this embodiment, the pressure means are formed in one piece with the protective cover 20. According to a variant embodiment, the pressure means may consist of an added part, such as for example a seal, arranged between the protective cover 20 and the door/flange seal 13. In the embodiment described, the annular boss is continuous. According to a variant embodiment, there may be provided a discontinuous boss forming distinct pressure points.

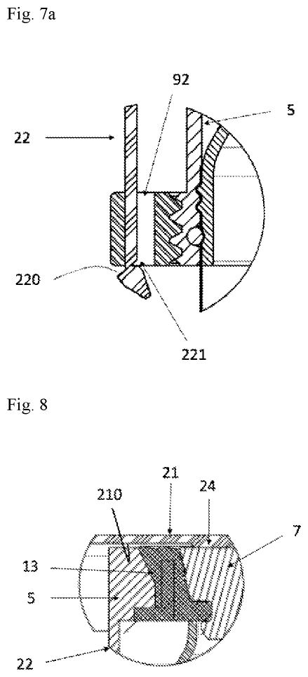

Advantageously, the tabs 22, 23 are arranged so that the protective cover 20 can be removed by deformation of said tabs. To do this, they each have a V-shaped notch arranged opposite the shoulder 220, 230. FIGS. 7 and 7a show a detail view of a tab thus arranged, only the tab 22 and its notch 221 being shown It could also be provided, according to a variant embodiment, for the tabs 22, 23 to be scored at the notches. Thus, depending on the material used, the embrittlement obtained via the notches allows either a localized deformation of the tabs 22, 23, or breaking of the tabs at the notch.

Due to the arrangement of its component elements, the connection device is assembled by simple clipping of the elements to one another. To make the container 1, the part of the flexible film located on the side of the through-opening will be arranged so as to be placed between the external sleeve 5 and the internal sleeve 6. The clamping will be effective by clamping the ring 9 on the internal sleeve 6, causing the tabs 12 to be gripped against the internal sleeve 6. The flexible film 2 may be disassembled by unscrewing the clamping ring 9 and unclipping the external sleeve 5 from the internal sleeve 6. The disassembly of the flexible film can only be carried out, however, after removal of the protective cover 20.

The flexible film 2 and the connection device are advantageously made of a bacteriostatic material. The seals 8 and 13 are made of a silicone material or other materials minimizing remanence phenomena. Provision may also be made for the seals to be made of a bacteriostatic material.

In the embodiment just described, the contact pressure means are arranged to exert contact pressure on the seal. According to a variant embodiment illustrated in FIG. 8, provision may be made for the contact pressure means to be arranged to exert contact pressure on the door 7. As described above, the pressure means can be formed by a continuous or discontinuous annular boss 24, provided at the internal face 210 of the covering part 21 and arranged to be placed opposite the door 7 when the protective cover 20 is placed on the container. They may also be borne by an added part (seal or other) arranged between the protective cover 20 and the door 7. In the embodiment described, the annular boss is continuous.

The invention is described in the foregoing by way of example. It is understood that a person skilled in the art is in a position to produce various variant embodiments of the invention without thereby departing from the scope of the invention.

1. A sealed container comprising a flange that delimits a through-opening having an axial axis in the container, a door movably mounted on the flange and a first annular seal located between the flange and the door, the flange, the door and the first seal forming a sealed connection device that can be connected to a complementary connection device on a sterile enclosure by bayonet connection means in order to allow sterile communication between the sealed container and the sterile enclosure, characterized in that the container comprises:
a protective cover on the connection device forcibly clipped onto the container; and
contact pressure means arranged between the protective cover and the connection device so as to apply contact pressure to the connection device in the axial direction proportional to the clipping force.

2. The sealed container according to claim 1, characterized in that the contact pressure means are arranged to exert contact pressure on the door.

3. The sealed container according to claim 1, characterized in that the contact pressure means are arranged to exert contact pressure on the first seal.

4. The sealed container according to claim 1, characterized in that the protective cover comprises an internal surface provided with an annular boss forming the pressure means.

5. The sealed container according to claim 4, characterized in that the annular boss is discontinuous.

6. The sealed container according to claim 1, characterized in that the pressure means are formed by a second annular seal inserted between the protective cover and the connection device.

7. The sealed container according to claim 1, characterized in that the sealed container has a container body formed by a flexible film fixed to the flange, said flange being made up of two parts that are separate from each other, respectively forming an external sleeve and an internal sleeve, the flexible film being kept engaged between the external sleeve and the internal sleeve.

8. The sealed container according to claim 7, characterized in that the sealed container comprises a ring for clamping the external sleeve on the internal sleeve arranged to grip the parts of the external sleeve and of the internal sleeve between which the flexible film is interposed.

9. The sealed container according to claim 8, characterized in that the protective cover comprises flexible tabs extending in the direction of the clamping ring and engaged with the latter in an irreversible manner.

10. The sealed container according to claim 5, characterized in that the sealed container comprises a third annular seal housed in an internal peripheral groove of the external sleeve, the flexible film extending between the annular seal and the internal sleeve.

11. The sealed container according to claim 1, characterized in that the external sleeve comprises a lower skirt comprising longitudinal slots extending from the lower end edge of the external sleeve and evenly distributed over the periphery of said sleeve so as to form elastic tabs capable of exerting a clamping force on the internal sleeve.

12. The sealed container according to any of the preceding claims, characterized in that the flexible film and the connection device are made of a bacteriostatic material.

13. The sealed container according to claim 6, characterized in that the sealed container comprises a third annular seal housed in an internal peripheral groove of the external sleeve, the flexible film extending between the annular seal and the internal sleeve.
